(19) United States
(12) Patent Application Publication
(10) Pub. No.: US 2026/0295201 A1
(43) Pub. Date: Oct. 1, 2026

(54) Portable Multisensory Wellness Kit

(71) Applicant: Prabhjot Kaur Sangra, Surrey (CA)

(72) Inventor: Prabhjot Kaur Sangra, Surrey (CA)

(21) Appl. No.: 19/540,555

(22) Filed: Feb. 13, 2026

Related U.S. Application Data
(60) Provisional application No. 63/780,458, filed on Mar. 31, 2025.

(51) Int. Cl. A61M 21/00; A61H 39/04

(52) U.S. Cl. CPC A61M 21/00; A61H 39/04; A61H 2201/102; A61M 2021/0016; A61M 2021/0022; A61M 2021/0044; A61M 2021/0066

(57) ABSTRACT

A portable multisensory wellness kit comprising a primary portable container and a plurality of coordinated sensory engagement components configured for general wellness use. The primary container houses tactile, thermal, olfactory, visual, and proprioceptive components including: a tactile acupressure device; a thermal biofeedback indicator providing a visible temperature-dependent appearance change; a breath pacing element having a single continuous, non-crossing tracing path; an aromatherapy inhalation device; a weighted anchoring object; an acupoint tapping instruction element; and instructional inserts. The kit further comprises a portable secondary container configured to selectively transport one or more of the sensory engagement components for on-the-go use. The components are configured for user-directed grounding, calming, and sensory engagement in non-medical general wellness contexts.

### CROSS-REFERENCE TO RELATED APPLICATIONS

This application claims priority to the U.S. Provisional Patent Application No. 63/780,458, filed Mar. 31, 2025, titled “Portable multisensory Regulation Kit with Biofeedback and Somatic Self-Soothing Tools for Nervous System Support and Emotional Relief,” the disclosure of which is incorporated herein by reference in its entirety.

### TECHNICAL FIELD

The present invention relates to consumer wellness and sensory support systems. More particularly, it relates to portable kits comprising multiple physical sensory engagement components configured to support grounding, calming, and user-directed sensory awareness through tactile, visual, olfactory, and proprioceptive interaction in general wellness contexts.

### BACKGROUND OF THE INVENTION

Many individuals seek accessible tools to support grounding, calming, or sensory organization during everyday activities. Existing consumer products in this space are typically single-modality in nature, such as isolated tactile objects, scent products, or visual prompts, and are not coordinated as an integrated system.

Users often assemble multiple separate items to create a personalized grounding or calming routine, resulting in inconsistency, inconvenience, and lack of portability. There remains a need for a compact, portable kit that integrates multiple sensory engagement elements into a unified system while allowing flexible, user-directed use without requiring medical diagnosis, treatment, or clinical supervision.

Even where individual sensory engagement elements are known in isolation, there remains a need for an integrated, portable kit architecture that coordinates tactile pressure stimulation, thermochromic visual feedback, paced breathing tracing, olfactory inhalation, rhythmic tapping guidance, and a weighted anchoring object within a unified general wellness system.

### SUMMARY OF THE INVENTION

The invention provides a portable multisensory wellness kit comprising a coordinated set of physical sensory engagement components housed within a portable container. The components are configured for user-directed grounding, calming, and sensory engagement through non-medical, general wellness interaction.

The kit includes:
a portable container;
a tactile acupressure device;
a thermal biofeedback indicator;
a breath pacing element;
an aromatherapy inhalation device;
a proprioceptive anchoring object;
an acupoint tapping instruction element; and
one or more instructional inserts describing component use.

The components may be used individually or in combination, in any order selected by the user, to support grounding, calming, or sensory awareness experiences. The kit is configured for general wellness sensory engagement and is not intended to diagnose, treat, cure, or prevent any disease or medical condition.

### BRIEF DESCRIPTION OF THE DRAWINGS

FIG. 1 is an exploded view of the complete kit showing a main container body, a lid, an upper tray, a lower tray, a representative instructional insert envelope, and component-receiving regions arranged in separated alignment.

FIG. 2 is a perspective view of the assembled kit in a closed configuration.

FIG. 3 is a top view of the upper tray configured to hold a plurality of instructional-insert envelopes, with one representative envelope shown for illustrative purposes.

FIG. 4 is a top view of the lower tray showing recesses configured to receive the sensory-engagement components.

FIG. 5 is an enlarged view of the breath pacing element showing a generally circular substrate having a single continuous, non-crossing tracing path.

FIG. 6 is an enlarged perspective view of a thermal biofeedback indicator sheet and a corresponding storage sleeve.

FIG. 7 is an enlarged front view of the acupoint tapping instruction element showing a neutral human silhouette and tapping-point markers.

FIG. 8 is an enlarged view of a portable secondary container.

FIG. 9 is an enlarged view of the proprioceptive anchoring object and an associated soft pouch.

FIG. 10 is an enlarged view of the aromatherapy inhalation device.

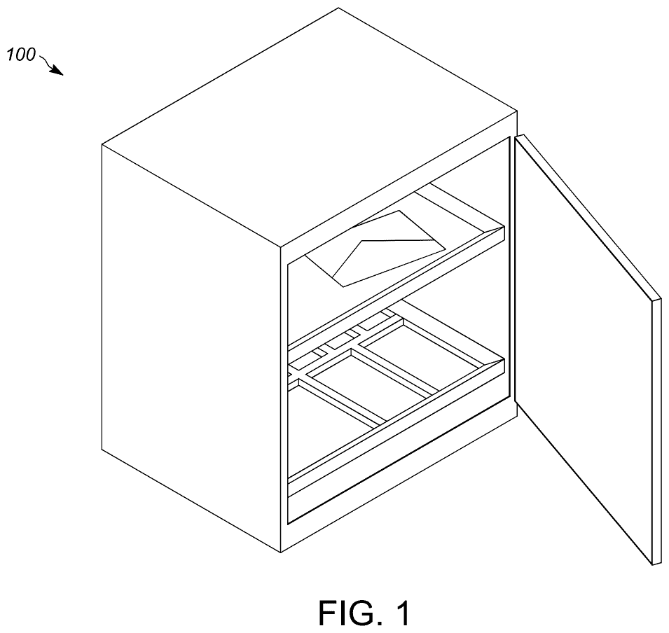

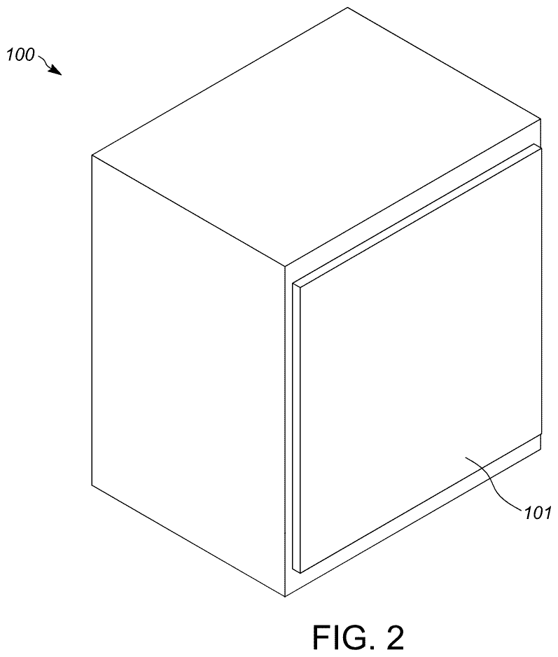

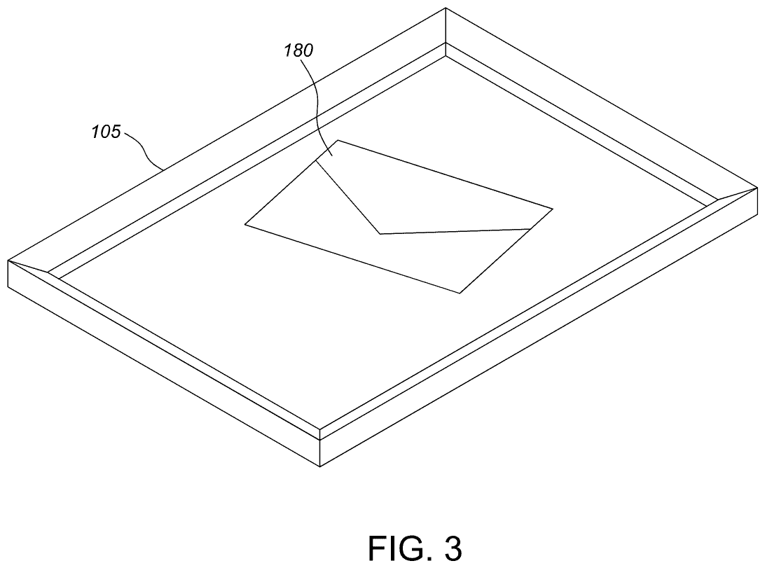

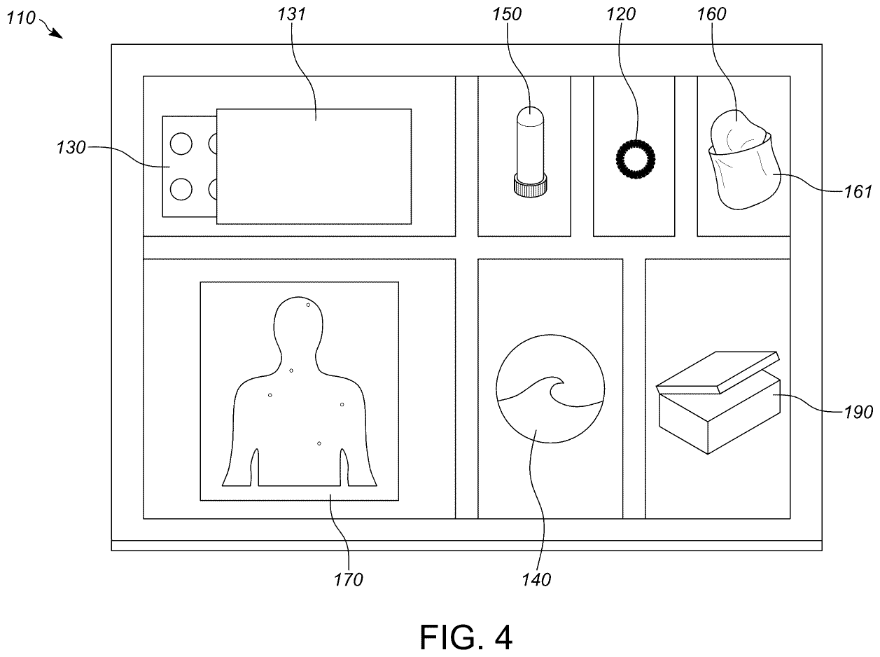

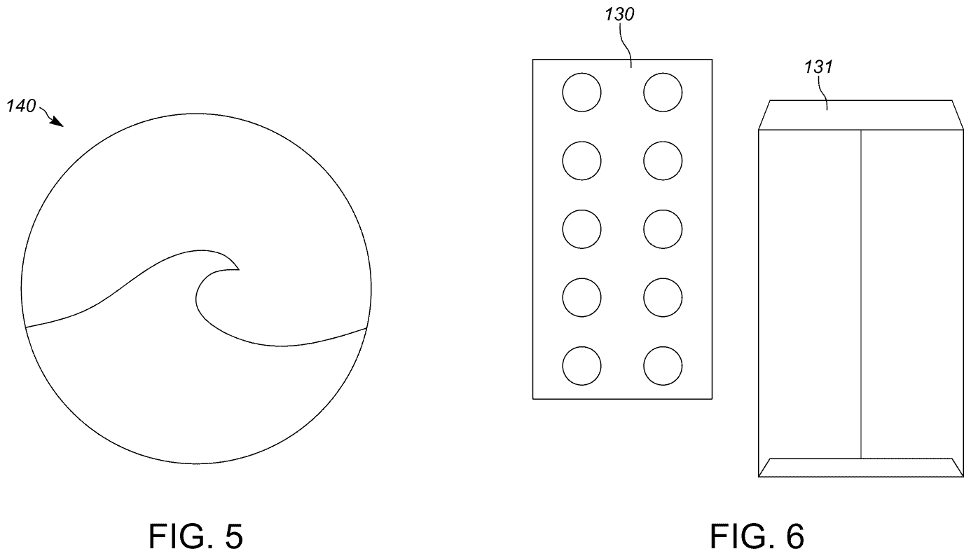

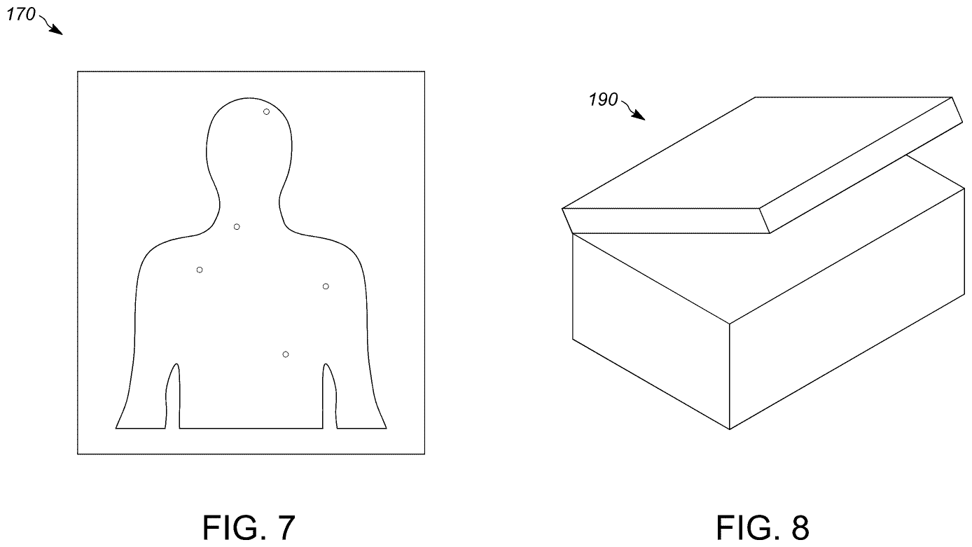

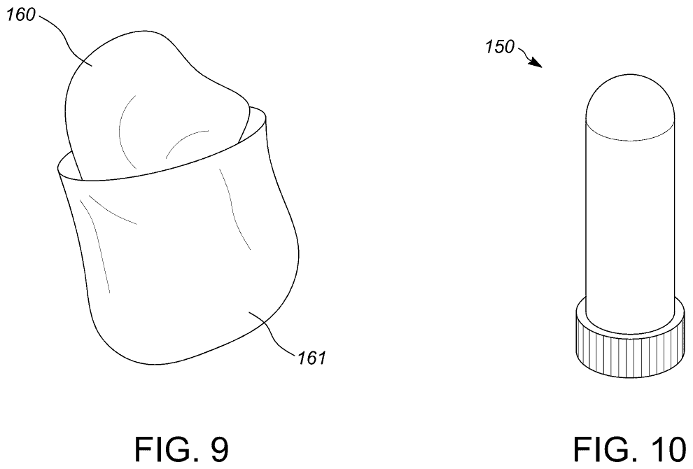

### DETAILED DESCRIPTION OF THE INVENTION

### Portable Container

The kit includes a portable container configured to store and organize the multisensory components. The container may include one or more internal trays, compartments, or recesses and may include a closure selected from hinged, sliding, removable, or equivalent access structures. Variations in container materials, geometry, or closure mechanisms do not limit the scope of the invention.

### Tactile Acupressure Device

The tactile acupressure device is a physical object configured to provide tactile pressure stimulation through user manipulation. The device may be formed as a closed-loop ring or similar structure and may include surface texturing, ridges, nodules, or compressive features to facilitate tactile engagement.

### Thermal Biofeedback Indicator

The thermal biofeedback indicator comprises a substrate including one or more thermochromic elements configured to provide a visible temperature-dependent appearance change when positioned against a user's skin or another surface contacting the body. The indicator may be provided in various shapes, quantities, or layouts, including dots, patches, or geometric forms. Observable appearance changes may include variations in darkness, lightness, hue, or pattern. Multiple indicators may be provided for repeated or extended use.

### Breath-Pacing Element

The breath-pacing element comprises a substrate having a single continuous, non-crossing tracing path formed as a wave-like or geometric pattern. The tracing path is configured to be visually followed or physically traced by a user's finger during paced breathing. Printed instructional content may optionally be included without defining functional performance.

### Aromatherapy Inhalation Device

The aromatherapy inhalation device comprises a body and a reservoir configured to contain one or more aromatic compounds for user inhalation. The device may be refillable or replaceable and is intended for sensory engagement in general wellness contexts.

### Proprioceptive Anchoring Object

The proprioceptive anchoring object is a weighted physical object configured to provide tactile and proprioceptive input when held, pressed, or manipulated. The object may be formed from stone, composite, or other materials and may be stored in a soft pouch.

### Acupoint Tapping Instruction Element

The acupoint tapping instruction element comprises a physical card including a neutral human silhouette and graphical tapping point markers. The tapping point markers are visual position indicators only and are not sensors, biofeedback elements, or therapeutic devices. Any textual content on the card is optional and informational in nature.

### Instructional Inserts

Instructional inserts may be provided to describe component use, sensory engagement suggestions, or grounding prompts. The inserts are informational only and do not constitute functional sensory components of the invention.

### METHOD OF USE

The components of the kit may be used individually or in combination, in any order selected by the user, to support grounding, calming, or sensory engagement experiences. Example use may include tactile interaction, observation of thermal appearance changes, paced breathing using the tracing path, olfactory inhalation, rhythmic tapping, and manipulation of the anchoring object. Steps may be omitted, rearranged, or repeated according to user preference.

1. A portable multisensory wellness kit comprising: a primary portable container configured to store a plurality of sensory engagement components; a tactile acupressure device configured to provide tactile pressure stimulation through user manipulation; a thermal biofeedback indicator comprising a substrate and at least one thermochromic element configured to provide a visible temperature-dependent appearance change when positioned against a user's skin or another surface contacting the body; a breath pacing element comprising a substrate having a single continuous, non-crossing tracing path configured to be visually followed or physically traced by a user during paced breathing; an aromatherapy inhalation device comprising a body and a reservoir configured to contain one or more aromatic compounds for user inhalation;
a proprioceptive anchoring object configured to provide weighted tactile input when held, pressed, or manipulated; an acupoint tapping instruction element comprising a physical card including a neutral human silhouette and graphical tapping point markers;
one or more instructional inserts describing use of at least one of the components; and
a portable secondary container configured to selectively receive and transport one or more of the sensory engagement components; wherein the components are configured for user-directed grounding, calming, and sensory engagement in general wellness contexts.

2. A multisensory wellness system comprising: a portable container defining an interior region; a plurality of physical sensory engagement components disposed within the interior region, the components including tactile, thermal, olfactory, visual, and proprioceptive modalities; and instructional media configured to facilitate user interaction with the physical components; wherein the system is configured for user-selected, non-medical general wellness use.

3. A method of using a portable multisensory wellness kit, the method comprising:
engaging a tactile acupressure device to provide tactile pressure stimulation; positioning a thermal biofeedback indicator against a user's skin or another surface contacting the body and observing a temperature-dependent appearance change; tracing a continuous path on a breath pacing element during paced breathing; inhaling aromatic compounds from an aromatherapy inhalation device; performing rhythmic tapping using an instruction element having graphical tapping-point markers; and holding, pressing, or otherwise manipulating a weighted anchoring object; wherein the steps may be performed in any order, combination, or repetition for general wellness sensory engagement.

4. The kit of claim 1, wherein the tactile acupressure device comprises a closed-loop ring.

5. The kit of claim 1, wherein the tactile acupressure device includes surface texturing, ridges, or nodules.

6. The kit of claim 1, wherein the thermochromic element comprises microencapsulated thermochromic pigment.

7. The kit of claim 1, wherein the thermal biofeedback indicator comprises a plurality of thermochromic elements arranged in a predetermined layout.

8. The kit of claim 1, wherein the breath pacing element comprises a raised tracing path.

9. The kit of claim 1, wherein the breath pacing element optionally includes printed instructional text.

10. The kit of claim 1, wherein the portable container comprises internal trays including an upper tray configured to hold instructional inserts and a lower tray configured to receive the sensory engagement components.

11. The kit of claim 1, wherein the proprioceptive anchoring object is stored in a soft pouch.

12. The kit of claim 1, wherein the graphical tapping-point markers are visual position indicators only and are not sensors or biofeedback elements.
